(19) United States
(12) Patent Application Publication
(10) Pub. No.: US 2013/0066299 A1
(43) Pub. Date: Mar. 14, 2013

(54) Wire Guide Grabbing Mechanism And Method

(71) Applicant: Thomas A. Kay, JR., Bloomington, IN (US)

(72) Inventor: Thomas A. Kay, JR., Bloomington, IN (US)

(73) Assignee: COOK MEDICAL TECHNOLOGIES LLC, Bloomington, IN (US)

(21) Appl. No.: 13/603,766

(22) Filed: Sep. 5, 2012

Related U.S. Application Data
(60) Provisional application No. 61/532,341, filed on Sep. 8, 2011.

(51) Int. Cl. A61M 25/09

(52) U.S. Cl. 604/528

(57) ABSTRACT

A wire guide grabbing mechanism includes an elongate mandrel having a proximal and distal mandrel end, and a grabber irreversibly attached to the mandrel. The grabber includes a plurality of struts arranged in a reticulated pattern and a plurality of openings defined by the plurality of struts. The grabber is adjustable between an expanded configuration at which the plurality of struts are spread out relative to one another and a contracted configuration at which the plurality of struts are condensed, such that a wire guide may be enmeshed by the grabber in the contracted configuration. Related methodology is also disclosed.

### TECHNICAL FIELD

The present disclosure relates generally to a wire guide grabbing mechanism, and relates more particularly to condensing a reticulated grabber about a wire guide such that the reticulated grabber enmeshes the wire guide.

### BACKGROUND

Angioplasty, stenting and other techniques are well known practices for treating obstructed vessels within the human anatomy. In a conventional approach, a catheter is advanced through an entry point in the patient's skin and slid over a wire guide to a desired location within the patient's vasculature. The balloon, stent, or other treatment device may be placed within or near an obstruction in the vessel of interest, and then used to increase or restore blood flow. Various techniques have been used with great success for decades. As with other forms of peripheral intervention, clinicians continue to seek the capability to treat smaller vessels and those located in more difficult to access places within the human body.

Advancements in medical device technology have allowed treatment devices to traverse relatively great lengths within the body and reach constrictions within especially small vessels. Approaching a treatment location may be of little use, however, unless the associated wire guide over which a treatment device travels is able to successfully cross a constriction to enable advancing the treatment device into or past the constriction. Those skilled in the art will be familiar with the relative difficulty of pushing a wire guide through material of a lesion blocking a vein or artery in many instances. In the case of treating infrapopliteal arteries, for instance, matters may be further complicated by the location and nature of the disease. A significant challenge for a treating physician can be crossing constricted areas in these vessels from a vascular access site that is relatively far away. In one conventional approach, a sheath is inserted retrograde to blood flow in the femoral artery in the leg opposite the one to be treated. The sheath and the wire guide are navigated up through the iliac, and then steered down into the opposite leg. The wire guide may eventually be advanced past the sheath through the diseased vessel of interest, such as the popliteal artery, the anterior tibial, posterior tibial or peroneal artery. Crossing lesions in the diseased vessel from such a distance access point may be quite difficult. Each twist and turn through the tortuous path navigated just to reach the diseased vessel can reduce pushability of the wire guide. Moreover, should the diseased vessel have a chronic total occlusion, the wire guide may need to punch through a fibrous thrombus cap at the distal end of the legion. These fibrous caps may be calcified and especially difficult to puncture given the conventional wire guide's atraumatic distal tip.

Alternative approaches attempt to access the vessel to be treated through the same leg femoral artery, anterograde to blood flow. This strategy enables a relatively straight approach and shorter distance to the lesion to be treated, however, the external anatomy of the patient may not be conducive to this type of technique. Moreover, while force transmission through the wire guide and steering may be easier, the challenge of crossing a fibrous thrombus cap at the distal end of the lesion is not significantly diminished.

A relatively newer technique for crossing challenging lesions involves accessing the diseased artery from the ankle or foot and traversing the lesion retrograde to blood flow. A wire guide introduced in this manner may be more readily capable of puncturing a fibrous cap at the distal end of the lesion given the fairly short, straight approach and direct access. As an alternative to puncturing the fibrous cap, the wire guide is sometimes taken subintimally and then reenters the vessel on the other side of the lesion. In either case, if the wire guide successfully crosses the lesion, it can be captured with a snare placed above the lesion, i.e. upstream, and then pulled out from the patient's body at an upstream entry point. Snaring the wire guide, however, is by no means certain using conventional techniques. Moreover, conventional snaring devices even theoretically capable of grabbing a wire guide under such circumstances tend to be complex and expensive.

### SUMMARY OF THE DISCLOSURE

In one aspect, a wire guide grabbing mechanism for use in percutaneously treating a patient includes an elongate mandrel having a proximal mandrel end, a distal mandrel end, and a grabber irreversibly attached to the mandrel and projecting from the distal mandrel end. The grabber includes a plurality of struts arranged in a reticulated pattern and a plurality of openings defined by the plurality of struts. The grabber has an expanded configuration at which the plurality of struts are spread out relative to one another, and a contracted configuration at which the plurality of struts are condensed, such that adjusting the grabber from the expanded configuration to the contracted configuration enmeshes a wire guide extending through at least one of the plurality of openings.

In another aspect, a method of percutaneously treating a patient includes advancing a wire guide through a body lumen of the patient from a first percutaneous entry point, and condensing a reticulated grabber about the wire guide such that the reticulated grabber enmeshes the wire guide. The method further includes withdrawing the reticulated grabber from a second percutaneous entry point to the body lumen while enmeshed with the wire guide, such that the wire guide simultaneously extends out of the patient at each of the first and second percutaneous entry points.

In still another aspect, a wire guide and grabbing mechanism assembly includes a wire guide, and a grabbing mechanism. The grabbing mechanism includes an elongate mandrel having a proximal mandrel end and a distal mandrel end, and a grabber irreversibly attached to the mandrel and projecting from the distal mandrel end. The grabber includes a plurality of struts arranged in a reticulated pattern, and a plurality of openings defined by the plurality of struts. The grabber includes an expanded configuration at which the plurality of struts are spread out relative to one another, and a contracted configuration at which the plurality of struts are condensed. The wire guide extends through at least one of the plurality of openings, and the grabbing mechanism is in the contracted configuration and enmeshes the wire guide.

### BRIEF DESCRIPTION OF THE DRAWINGS

FIG. 1 is a partially sectioned side diagrammatic view of a treatment system according to one embodiment;

FIG. 2 is a side diagrammatic view of a wire guide grabbing mechanism according to one embodiment;

FIG. 3 is an end view of the wire guide grabbing mechanism of FIG. 2;

FIG. 4 is a side diagrammatic view of a wire guide grabbing mechanism according to another embodiment;

FIG. 5 is a side diagrammatic view of a wire guide grabbing mechanism according to yet another embodiment;

FIG. 6 is a diagrammatic view of one stage of a treatment procedure;

FIG. 7 is a diagrammatic view of another stage of the treatment procedure;

FIG. 8 is a diagrammatic view of yet another stage of the treatment procedure; and

FIG. 9 is a diagrammatic view of yet another stage of the treatment procedure.

### DETAILED DESCRIPTION

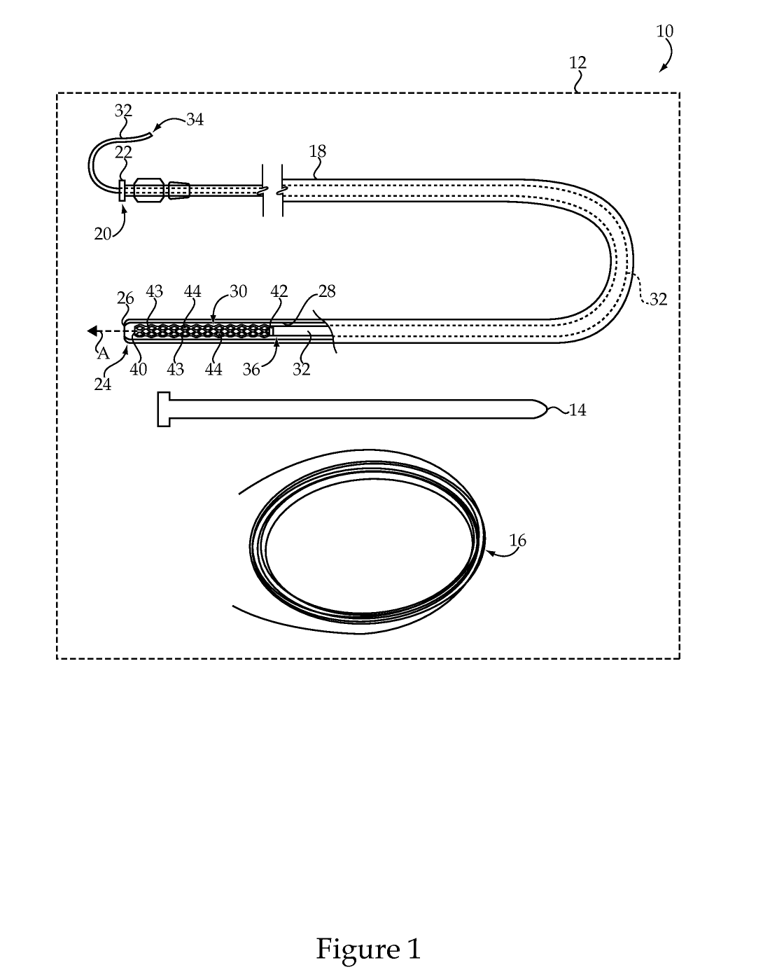

Referring to FIG. 1, there are shown components of a treatment system 10 according to one embodiment, placed within a sterile, peel-open package 12. System 10 is depicted as including a wire guide 16, an introducer 14, and a wire guide grabbing mechanism 30 positioned within a sheath 18. In other embodiments, mechanism 30 and sheath 18 might be provided to an end user by themselves, or even mechanism 30 by itself, and other components of system 10 obtained from supplies ordinarily kept on hand. As will be further apparent from the following description, mechanism 30 is contemplated to provide an advantageous means for grasping wire guide 16 when fed into a body lumen of a patient from a first percutaneous entry point, such that wire guide 16 may be pulled out of the patient at a second percutaneous entry point, to enable subsequent steps in a treatment procedure.

As noted above, mechanism 30 may be positioned within sheath 18 for shipment and storage, although the present disclosure is not thereby limited. Sheath 18 may include a proximal end 20, and a distal end 24. A fitting or manifold 22 may be located at proximal end 20, and an opening 26 for deploying mechanism 30 as further described herein may be formed in distal end 24. A lumen 28 extends longitudinally through sheath 18 from proximal end 20 to distal end 24, and mechanism 30 is positioned within lumen 28.

Wire guide grabbing mechanism 30 may include an elongate mandrel 32 having a proximal mandrel end 34, shown extending in a proximal direction from fitting 22. Mandrel 32 may also include a distal mandrel end 36 shown positioned inside of sheath 18 slightly proximal of distal end 24. Mechanism 30 may further include a grabber 40 irreversibly attached to mandrel 32 via a coupling 42. Coupling 42 may include a welded connection or another type of connection, but will not be intended nor configured to enable grabber 40 to be disconnected from mandrel 32. Disconnecting grabber 40 from mandrel 32 would permanently damage mechanism 30, and render it unusable for the purposes contemplated herein. Grabber 40 may be oriented such that it projects from distal mandrel end 36, and includes a plurality of struts 43, arranged in a reticulated pattern, and a plurality of openings 44 defined by struts 43.

Grabber 40 may further include an expanded configuration at which struts 43 are spread out relative to one another, and a contracted configuration at which struts 43 are condensed. In the expanded configuration, struts 43 may be understood to be spaced from one another in two dimensions or three dimensions at relatively greater distance, whereas in the contracted configurations struts 43 may be understood to be spaced from one another in two dimensions or three dimensions at relatively lesser distance. In a manner further described herein, adjusting grabber 40 from the expanded configuration to the contracted configuration can enmesh a wire guide extending through at least one of openings 44. In one embodiment, grabber 40 may be self-expanding such that grabber 40 assumes its expanded configuration in response to relieving a condensing bias thereon. In FIG. 1, grabber 40 is shown as it might appear where a condensing bias of sheath 18 is applied such that grabber 40 is held in its contracted configuration within sheath 18. It will be further understood that grabber 40 could be characterized as having a range of states from a fully contracted state, at which struts 43 are condensed such that essentially no spacing exists between adjacent struts, to a fully expanded state sat which zero condensing bias is applied and struts 43 are spread out as far as possible without deforming or breaking “Condensed” and “expanded” are thus used herein in a relative sense.

Grabber 40 further defines a longitudinal axis A, which may be collinear with a longitudinal center axis of mandrel 32. In one embodiment, the expanded configuration may include a radially expanded configuration, whereas the contracted configuration may include a radially contracted configuration, relative to longitudinal axis A. Accordingly, when grabbing mechanism 30 is slid axially relative to sheath 18 such that grabber 40 extends out of opening 26, portions of grabber 40 which are not within lumen 28 may radially expand outwardly from longitudinal axis A by way of an inherent self-expanding bias of grabber 40. Grabber 40 might additionally or alternatively axially lengthen when adjusted from its contracted configuration to its expanded configuration, or vice versa. Each of mandrel 32 and grabber 40 may be formed from metallic materials. Grabber 40 may be formed from stainless steel, nickel-tin alloy, palladium or any other metallic material capable of being manufactured to have suitable shape memory properties, such that grabber 40 may spring outwardly when a condensing bias of sheath 18 is reduced or removed, as further described herein. Materials from which grabber 40 is made, or with which it is coated, are desirably radiopaque.

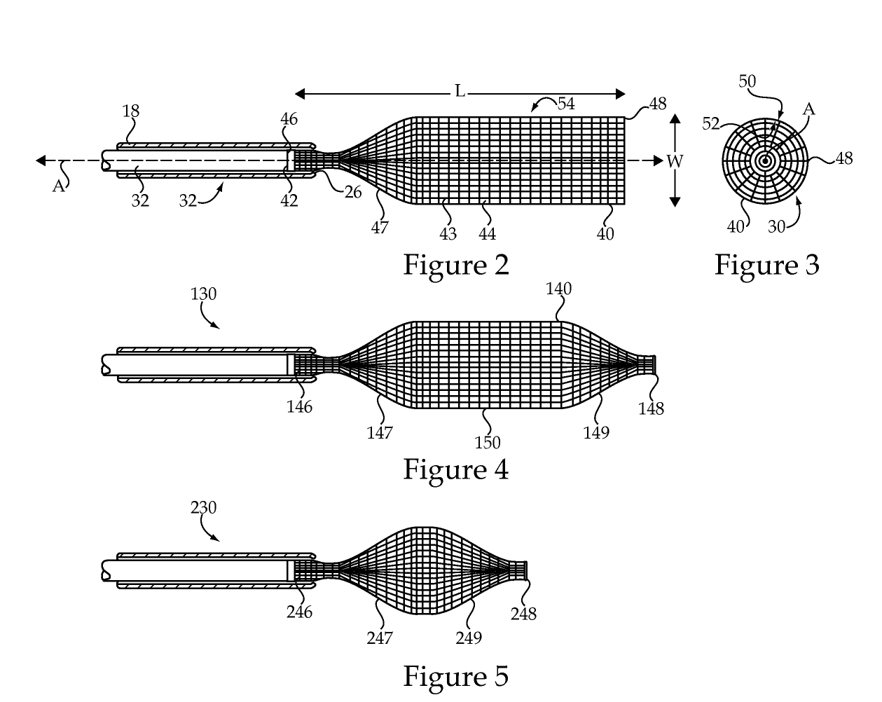

Turning now to FIG. 2, there is shown a side diagrammatic view of grabber 40 and a portion of mandrel 32, as grabber 40 might appear when slid out of sheath 18 to assume its expanded configuration. As shown in FIG. 2, an axial grabber length L extends from a proximal grabber end 46 to a distal grabber end 48, and a radial grabber width W extends in a direction normal to longitudinal axis A. In one practical implementation strategy, width W may be less than length L, for instance by a factor of three or more. In the expanded configuration of grabber 40, width W may increase by a factor of 1.5 or more as compared with width W in the contracted configuration, although of course this difference would depend upon how tightly grabber 40 is condensed by sheath 18, and whether grabber 40 is restricted from fully expanding such as by contacting an inner wall of a body lumen of a patient, as further described herein.

Referring also to FIG. 3, illustrating an end view of grabber 40, it may be noted that struts 43 define an enclosure having a regular shape extending circumferentially about longitudinal axis A. In particular, from FIG. 3 it may be noted that an outer periphery 50 of grabber 40 defines a cylindrical shape, whereas an inner periphery 52 also defines a cylindrical shape. FIG. 3 may thus be understood as a view into an interior space defined by struts 43, the interior space being substantially free of struts 43. As discussed above, adjusting grabber 40 from the expanded configuration to the contracted configuration can enmesh a wire guide passed through at least one of openings 44. In FIG. 2, grabber 40 may be understood as having the form generally of a one-piece screen body, consisting essentially of struts 44. A taper 47 is located adjacent to proximal grabber end 46, and transitions to a cylinder extending in a distal direction from taper 47 to distal end 48. A side 54 of grabber 40 extends between proximal grabber end 46 and distal grabber end 48. As further described herein, in one practical implementation strategy the wire guide to be enmeshed by adjusting grabber 40 to its contracted configuration may be passed through one of openings 44 in side 54, and in particular passed through the cylindrical shaped portion of grabber 40 such that the end of the wire guide is located in or even passes all the way through the enclosure defined by struts 43. In the FIG. 2 embodiment, distal grabber end 48 may be open, such that the enclosure is a partial enclosure.

In one embodiment, a number of openings 44 may be about 20 or greater, and may be about 30 or greater. As used herein, the term “about” should be understood in the context of a number of significant digits. Thus, “about 20” means from 15 to 24, and “about 30” means from 25 to 34. In still further embodiments, a number of openings 44 might be about 100, or still greater. As noted above, openings 44 are defined by struts 43, and may have shapes and sizes which result from the particular reticulated pattern chosen in constructing grabber 40. Each of struts 43 may be irreversibly attached to at least one other of struts 43, and in certain embodiments each of struts 43 might be understood to be directly and irreversibly attached to four, six, or more other struts.

As noted above, grabber 40 may have the general form of a screen such as might be fashioned by attaching a plurality of different wires. While FIG. 2 is meant to be understood as diagrammatic only, it may be noted that a plurality of struts 43 in grabber 40 run generally lengthwise, whereas another plurality of struts 43 are arranged generally circumferentially. Attaching a plurality of longitudinal wires to a plurality of circumferential wires at at least some of the locations where the sets of wires overlap could render a configuration similar to that shown in FIG. 2. A plurality of individual wires might also be woven, knitted or coupled together in some other fashion analogous to techniques used in the field of textile engineering. Another strategy for forming grabber 40, for example as a one-piece body consisting essentially of struts 43, is machining or laser cutting openings 44 in a blank. For instance, a metallic blank may be provided and material removed from the metallic blank to render a one-piece body comprised of nothing but struts 43. Versions are also contemplated where a fluropolymer coating or the like is applied to grabber 40. Such an embodiment could still be understood as consisting essentially of struts 43. As discussed above, in the embodiment of FIG. 2 taper 47 may comprise an axial segment of grabber 40, whereas the portion of grabber 40 extending from taper 47 to distal end 48 comprises a non-tapered segment, which may be cylindrical. Alternative configurations are also contemplated herein.

Referring now to FIG. 4, there is shown a wire guide grabbing mechanism 130 according to another embodiment, and having a grabber 140 which includes a proximal grabber end 146 and a distal grabber end 148. Grabber 140 includes a proximal taper 147, a distal taper 149, and a non-tapered segment 150 extending between taper 147 and taper 149. Segment 150 might include a cylindrical segment, whereas each of tapers 147 and 149 may be conical, or generally so. Distal grabber end 148 may include a closed end, at least relative to the open end 48 of the FIG. 2 embodiment. Referring now to FIG. 5, there is shown yet another wire guide grabbing mechanism 230, including a grabber 240 having a proximal grabber end 246 and a distal grabber end 248. In contrast to the previously described embodiment, grabber 240 includes adjoining proximal and distal tapers 247 and 249, respectively. A variety of other configurations are also contemplated herein, such as spherical or hemispherical grabbers, grabbers having non-circular axial cross sections, and barrel-shaped or funnel-shaped grabbers. Grabbers according to the present disclosure might also include polygonal axial cross sections or even irregular cross sections. It may further be noted that each of the embodiments discussed in connection with FIGS. 2-5 defines a longitudinal axis which is substantially straight. In other embodiments, an S-shaped longitudinal axis, an angular longitudinal axis, or still some other shape might be used.

### INDUSTRIAL APPLICABILITY

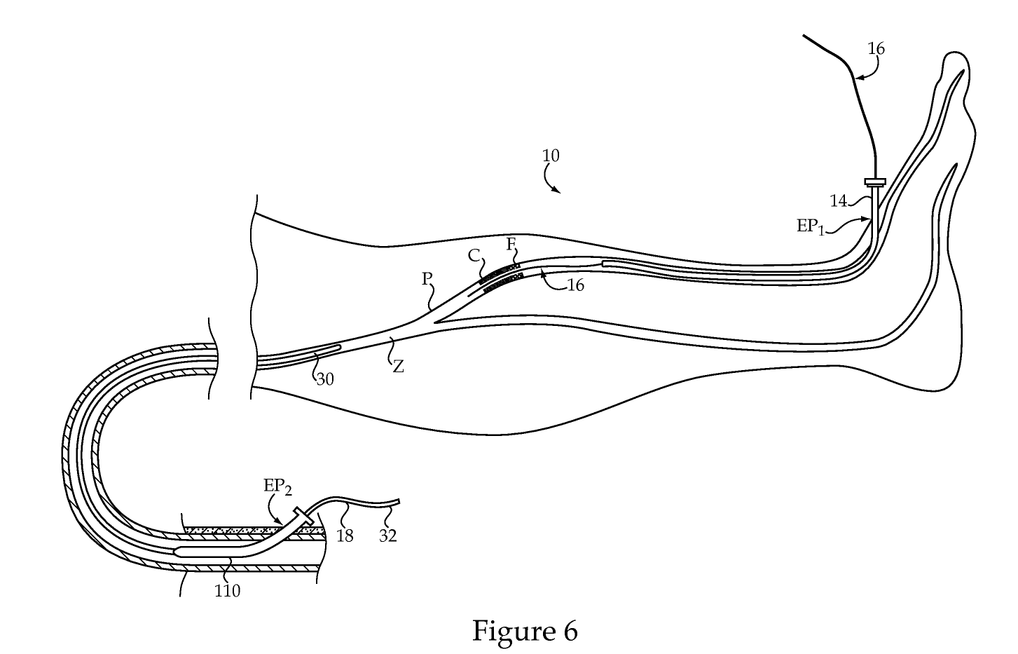

Referring now to FIG. 6, there is shown system 10 as it might appear at one stage of performing a percutaneous treatment procedure on a patient. Introducer 14 extends into a body lumen P of the patient at a first percutaneous entry point EP₁, below the patient's knee and in the patient's foot, for example. Entry point EP₁ might instead be in the patient's ankle In one embodiment, introducer 14 may extend into the pedis dorsalis artery. Wire guide 16 has been advanced through introducer 14 and through the body lumen in an upstream or retrograde direction from first percutaneous entry point EP₁, and is shown as it might appear having just crossed a constriction C within body lumen P. As discussed above, constrictions in the human vasculature may include a fibrous thrombus cap which can be challenging to cross via conventional anterograde access. Accordingly, wire guide 16 may include a suitable constriction crossing wire which has been advanced in the upstream or retrograde direction, i.e. toward the patient's heart, to punch through a fibrous thrombus cap F of constriction C. An access wire, softer than wire 16, may be used in establishing entry point EP₁, and then swapped for wire 16, in a generally known manner.

Wire guide grabbing mechanism 30 extends through a second introducer 110 extending into a body lumen Z at a second percutaneous entry point EP₂. In one embodiment, entry point EP₂ may include an entry point to the contralateral femoral artery. Mechanism 30 is shown as it might appear having been advanced in a retrograde direction through the contralateral femoral artery, and then an anterograde direction through the same leg femoral artery, and eventually reaching a location just upstream of the artery containing constriction C. Mandrel 32 is shown positioned outside the patient's body, and extending in a proximal direction from sheath 18.

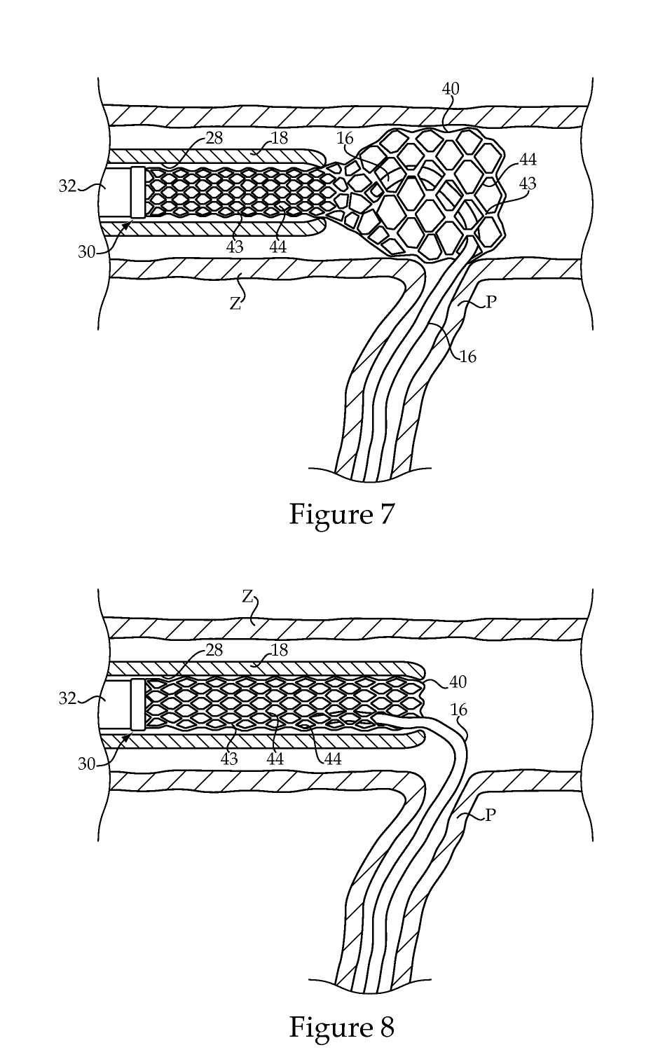

From the stage depicted in FIG. 6, mechanism 30 may be further advanced in an anterograde direction such that grabber 40 may be deployed approximately at an intersection of lumen Z and lumen P. Referring now to FIG. 7, there is shown mechanism 30 as it might appear where sheath 18 has been withdrawn relative to grabber 40, allowing part of grabber 40 to assume its expanded configuration. In particular, removing the condensing bias of sheath 18 may allow grabber 40 to expand to or towards its expanded configuration, spreading out struts 43 within body lumen Z. In one practical implementation strategy, during deploying grabber 40 sheath 18 may be advanced past a point at which lumen P intersects with lumen Z, and then withdrawn such that grabber 40 remains essentially stationary but for its expansion radially outwardly and into contact with inner walls of body lumen Z.

During or subsequent to expanding grabber 40, wire guide 16 may be advanced through body lumen P such that wire guide 16 passes through one of openings 44, and enters into the interior of grabber 40. Once wire guide 16 passes into one of openings 40, grabber 40 or sheath 18 may be moved upstream or downstream slightly to assist in directing the end of wire guide 16 deeper into or through the interior of grabber 40. In FIG. 7, grabber 40 is shown only partially deployed, such that approximately a proximal half of grabber 40 remains condensed within sheath 18. In other embodiments, it may be desirable and/or necessary to deploy relatively more or relatively less of grabber 40 within the body lumen. It is believed that contact of grabber 40 with inner walls of the body lumen can assist in holding grabber 40 relatively stationary, while wire 16 is fed through one of openings 44. It is also contemplated that wire guide 16 may be fed completely through grabber 44, or grabber 44 itself manipulated within body lumen Z, such that wire guide 16 ultimately passes through more than one of openings 44.

Referring now to FIG. 7, there is shown mechanism 30 as it might appear after feeding wire guide 16 through one of openings 44, and where grabber 40 has been condensed while wire guide 16 is positioned within the one of openings 44. Accordingly, it will be understood that grabber 44 has been condensed about wire guide 16, such that grabber 40 enmeshes wire guide 16. To reach the stage depicted in FIG. 8, sheath 18 may be advanced over grabber 40 such that a condensing bias of sheath 18 is applied to grabber 40, and condenses grabber 40 in opposition to its self-expanding bias. In the condensed state of grabber 40, with wire guide 16 enmeshed, a number of struts 43 greater than two may contact wire guide 16. In many instances, condensing grabber 40 may result in many struts 43 contacting wire guide 16, such that wire guide 16 is coupled with grabber 40 by way of frictionally interacting with wire guide 16 at five, ten, or an even greater number of points of contact. It may also be noted from the FIG. 7 and FIG. 8 illustrations that openings 44 have a different shape when grabber 40 is in its expanded configuration, versus the contracted configuration. In one practical implementation strategy, each of openings 44 may define a fixed size and a mutable shape. In FIG. 7, it may be noted that openings 44 within the portion of grabber 40 resident in sheath 18 are relatively elongate, whereas openings 44 in the part of grabber 40 outside of sheath 18 are relatively less elongate, or not at all. In light of this, it will be understood that condensing grabber 40 may squeeze struts 43 relatively close together such that a shape of openings 44 changes, in some instances pinching wire guide 16 between struts defining the one or more openings 44 through which wire guide 16 passes.

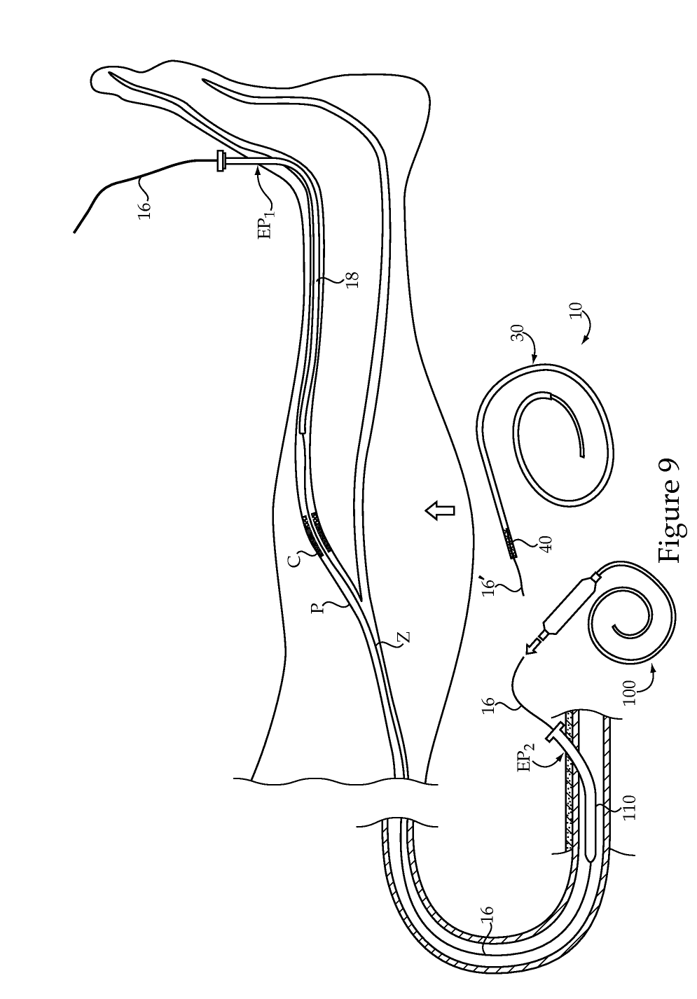

Referring now also to FIG. 9, upon condensing grabber 40 about wire guide 16 and enmeshing wire guide 16, mechanism 30 may be withdrawn from the second percutaneous entry point EP₂ such that wire guide 16 simultaneously extends out of the patient at each of the first and second percutaneous entry points. In FIG. 9, wire guide 16 has been cut, such that one end of the original wire guide 16 remains enmeshed with grabber 40 within mechanism 30, that portion of wire guide 16 being denoted via reference numeral 16′. In FIG. 9, grabber 40 may be understood as having been decoupled from wire guide 16, since the piece 16′ of original wire guide 16 will typically be so small as to no longer practically function as a wire guide. Rather than cutting wire guide 16, however, its enmeshment with grabber 40 might simply be reversed. Also shown in FIG. 9 is an intraluminal treatment mechanism comprising a conventional balloon catheter 100 positioned as it might appear just prior to commencing sliding balloon catheter 100 from entry point EP₂ over wire guide 16, through body lumen Z in a downstream or anterograde direction to ultimately guide catheter 100 into constriction C, or to another target location. Constriction C may then be dilated via balloon catheter 100 in a conventional manner, and appropriate activities under taken to remove wire guide 16 and catheter 100, and conclude the procedure. Catheter 100 might be configured to deploy a stent within constriction C₁, in addition to or as an alternative to performing angioplasty.

The present description is for illustrative purposes only, and should not be construed to narrow the breadth of the present disclosure in any way. Thus, those skilled in the art will appreciate that various modifications might be made to the presently disclosed embodiments without departing from the full and fair scope and spirit of the present disclosure. Other aspects, features and advantages will be apparent upon an examination of the attached drawings and appended claims.

What is claimed is:

1. A wire guide grabbing mechanism for use in percutaneously treating a patient comprising:
an elongate mandrel having a proximal mandrel end and a distal mandrel end; and
a grabber irreversibly attached to the mandrel and projecting from the distal mandrel end, the grabber having a plurality of struts arranged in a reticulated pattern and a plurality of openings defined by the plurality of struts;
the grabber having an expanded configuration at which the plurality of struts are spread out relative to one another, and a contracted configuration at which the plurality of struts are condensed, such that adjusting the grabber from the expanded configuration to the contracted configuration enmeshes a wire guide extending through at least one of the plurality of openings.

2. The mechanism of claim 1 wherein the grabber defines a longitudinal axis and the plurality of struts define an enclosure having a regular shape extending circumferentially about the longitudinal axis.

3. The mechanism of claim 2 wherein the grabber is self-expanding such that the grabber assumes the expanded configuration in response to relieving a condensing bias thereon.

4. The mechanism of claim 2 wherein the grabber has a length extending between a proximal grabber end and a distal grabber end, and a width in a direction normal to the longitudinal axis which is less than the length, and wherein the elongate mandrel is coaxial with the grabber.

5. The mechanism of claim 2 wherein the expanded configuration is a radially expanded configuration and the contracted configuration is a radially contracted configuration, relative to the longitudinal axis.

6. The mechanism of claim 5 wherein a number of the openings is about 20 or greater, and at least one of a size and a shape of the openings changes in response to adjusting the grabber between the expanded and contracted configurations.

7. The mechanism of claim 6 wherein a number of the openings is about 30 or greater, and wherein each of the openings defines a fixed size and a mutable shape.

8. The mechanism of claim 6 wherein each of the struts is irreversibly attached to at least one other of the plurality of struts, and is formed from a metallic material.

9. The mechanism of claim 6 wherein the grabber is formed as a one-piece body and consists essentially of the plurality of struts.

10. The mechanism of claim 5 wherein the grabber further includes a proximal grabber end and a distal grabber end, wherein at least one of the proximal and distal grabber ends includes a taper, and wherein the grabber further includes a non-tapered segment extending between the proximal and distal grabber ends.

11. A method of percutaneously treating a patient comprising the steps of:
advancing a wire guide through a body lumen of the patient from a first percutaneous entry point;
condensing a reticulated grabber about the wire guide such that the reticulated grabber enmeshes the wire guide; and
withdrawing the reticulated grabber from a second percutaneous entry point to the body lumen while enmeshed with the wire guide, such that the wire guide simultaneously extends out of the patient at each of the first and second percutaneous entry points.

12. The method of claim 11 further comprising a step of spreading out a plurality of struts of the reticulated grabber within the body lumen, prior to the step of condensing.

13. The method of claim 12 wherein the step of spreading out further includes spreading out the plurality of struts during expanding the grabber such that the grabber contacts an inner wall of the body lumen.

14. The method of claim 12 further comprising a step of feeding the wire guide through an opening in a side of the reticulated grabber, and wherein the step of condensing further includes condensing the grabber while the wire guide is positioned within the opening.

15. The method of claim 14 wherein the step of spreading out further includes withdrawing a sheath relative to the reticulated grabber, and the step of condensing further includes applying a condensing bias of the sheath to the reticulated grabber via advancing the sheath over the reticulated grabber.

16. The method of claim 15 wherein the step of condensing further includes condensing the reticulated grabber such that a number of the struts greater than two contacts the wire guide.

17. The method of claim 11 further comprising a step of decoupling the reticulated grabber from the wire guide at a location outside the patient, and guiding another intraluminal mechanism into the body lumen via the wire guide.

18. The method of claim 17 wherein the step of advancing further includes advancing the wire guide in an upstream direction from a first percutaneous entry point below the patient's knee, and wherein the step of guiding further includes guiding the intraluminal mechanism in a downstream direction to a target location within the body lumen from the second percutaneous entry point.

19. A wire guide and grabbing mechanism assembly comprising:
a wire guide;
a grabbing mechanism including an elongate mandrel having a proximal mandrel end and a distal mandrel end, and a grabber irreversibly attached to the mandrel and projecting from the distal mandrel end, the grabber having a plurality of struts arranged in a reticulated pattern, and a plurality of openings defined by the plurality of struts;
the grabber having an expanded configuration at which the plurality of struts are spread out relative to one another, and a contracted configuration at which the plurality of struts are condensed; and
wherein the wire guide extends through at least one of the plurality of openings, and the grabbing mechanism is in the contracted configuration and enmeshes the wire guide.

20. The assembly of claim 19 further comprising a sheath, wherein the grabbing mechanism is self-expanding and the sheath is positioned about the grabbing mechanism and the wire guide and applies a condensing bias to the wire guide such that the grabbing mechanism is held in the contracted configuration.
